(19) United States
(12) Patent Application Publication
(10) Pub. No.: US 2020/0002441 A1
(43) Pub. Date: Jan. 2, 2020

(54) METHOD OF CROSSLINKING GLYCOSAMINOGLYCANS

(71) Applicants: Galderma Research & Development; CENTRE NATIONAL DE LA RECHERCHE SCIENTIFIQUE

(72) Inventors: Rachel AUZELY-VELTY, Le Gua (FR); Tamiris FIGUEIREDO, Saint Martin d'Hères (FR); Laura JING JING, Antibes (FR); Craig Steven HARRIS, Biot (FR); Jean-Guy BOITEAU, Valbonne (FR); Thibaut GERFAUD, Mouans Sartoux (FR); Loic TOMAS, Biot (FR)

(73) Assignees: Galderma Research & Development, Biot (FR); CENTRE NATIONAL DE LA RECHERCHE SCIENTIFIQUE, Paris (FR)

(21) Appl. No.: 16/322,653

(22) PCT Filed: Aug. 2, 2017
(86) PCT No.: PCT/EP2017/069575

§ 371 Date: Feb. 1, 2019

Related U.S. Application Data
(60) Provisional application No. 62/370,479, filed on Aug. 3, 2016.

(30) Foreign Application Priority Data
Dec. 23, 2016 (EP) 16206622.9
Dec. 23, 2016 (EP) 16206624.5

(51) Int. Cl. C08B 37/08; A61K 8/73; A61Q 19/08

(52) U.S. Cl. CPC C08B 37/0072; A61Q 19/08; A61K 8/735

(57) ABSTRACT

A new hydrogel made of double crosslinked glycosaminoglycans, particularly crosslinked hyaluronic acid, chondroitin or chondroitin sulfate, having reversible linkages using boronic acid or boroxole derivatives leading to new benefits. Double crosslinked glycosaminoglycans, one linkage via two ether bonds with a hydroxyl group of each of two glycosaminoglycans and another linkage via an alkoxyboronate ester anion formed between a boronate hemiester grafted to one of the glycosaminoglycans and a diol function of to the other glycosaminoglycan. The diol function may be a backbone diol function or a diol portion of a diol functional moiety grafted the other glycosaminoglycan.

### TECHNICAL FIELD OF THE INVENTION

The invention relates to glycosaminoglycans crosslinked by a first and a second linkage, wherein the first linkage comprises two ether bonds and the second linkage is via an alkoxyboronate ester anion as well as a method for producing the same. The invention further relates to the use of a boronate hemiester in the manufacture glycosaminoglycans crosslinked by a first and a second linkage, wherein the first linkage comprises two ether bonds and the second linkage is via an alkoxyboronate ester anion.

### BACKGROUND OF THE INVENTION

Water-absorbing gels, or hydrogels, are widely used in the biomedical field. They are generally prepared by chemical crosslinking of polymers to infinite networks. While many polysaccharides absorb water until they are completely dissolved, crosslinked gels of the same polysaccharides can typically absorb a certain amount of water until they are saturated, i.e. they have a finite liquid retention capacity, or swelling degree.

Hyaluronic acid, chondroitin and chondroitin sulfate are well-known biocompatible polymers. They are naturally occurring polysaccharides belonging to the group of glycosaminoglycans (GAGs). All glycosaminoglycans are negatively charged heteropolysaccharide chains which have a capacity to absorb large amounts of water.

Hyaluronic acid (HA) is one of the most widely used biocompatible polymers for medical and cosmetic use. Hyaluronic acid and products derived from hyaluronic acid are widely used in the biomedical and cosmetic fields, for instance during viscosurgery and as a dermal filler.

Chondroitin sulfate (CS) is a highly abundant GAG found in the connective tissues of mammals where it, together with other sulfated GAGs, is bound to proteins as part proteoglycans. It has previously been shown that hydrogels containing CS successfully can be used in biomedical applications due to their resemblance to the natural extra cellular matrix (Lauder, R. M., Complement Ther Med 17: 56-62, 2009). Chondroitin sulfate is also used in the treatment of osteoarthritis, e.g. as a dietary supplement.

Crosslinking of the glycosaminoglycans prolongs the duration of the degradable polymers that make up the network, which is useful in many applications.

However, one of the main drawbacks of a large majority of the glycosaminoglycans-based gels, such as when used for treating wrinkles lies in the difficulty of injecting the hydrogel due to the high crosslinking density of the polysaccharide.

Hyaluronic acid is one of the most widely used biocompatible polymers for medical use. Hyaluronic acid and the other GAGs are negatively charged heteropolysaccharide chains which have a capacity to absorb large amounts of water. Hyaluronic acid and products derived from hyaluronic acid are widely used in the biomedical and cosmetic fields, for instance during viscosurgery and as a dermal filler.

Since hyaluronic acid is present with identical chemical structure except for its molecular mass in most living organisms, it gives a minimum of foreign body reactions and allows for advanced medical uses. Crosslinking and/or other modifications of the hyaluronic acid molecule is typically necessary to improve its duration in vivo. Furthermore, such modifications affect the liquid retention capacity of the hyaluronic acid molecule. As a consequence thereof, hyaluronic acid has been the subject of many modification attempts.

In the prior art, the hydrogels are prepared by reacting hyaluronic acid, for example, with BDDE (butanediol diglycidyl ether) in a basic aqueous medium resulting in the formation of covalent linkages (WO 97/04012). This is not a reversible process. WO 2014/072330 discloses a polymer composition comprising a mixture of phenylboronic acid modified hyaluronic acid polymer grafted on at least a hydroxyl with a group comprising phenylboronic acid and a cis-diol modified HA polymer grafted on at least a hydroxyl with a group comprising a cis-diol. WO 98/02204 discloses medical devices ionically and non-ionically crosslinked polymer hydrogels having improved mechanical properties. US 2014/0155305 discloses an aqueous solution comprising a thickening polymer with diol groups distributed along it, such as guar or other polysaccharide, which is cross linked with a cross-linker which contains a plurality of boroxole groups. US 2013/0129797 A1 discloses polymeric compositions that comprise at least one polymer residue and at least one crosslinking moiety, wherein the polymer residue is crosslinked by the crosslinking moiety and wherein the crosslinking moiety is formed from a reaction between a boronic acid moiety and a hydroxamic acid moiety.

### DESCRIPTION OF THE INVENTION

It is an object of the present invention to provide a hydrogel having a glycosaminoglycan (GAG) as the swellable polymer, having reversible linkages.

It is also an object of the invention to provide a self-healing and stable gel.

One object of the present invention to provide a method for preparing hydrogels of glycosaminoglycan molecules by mild and efficient routes.

Yet another object of the invention is to mitigate, alleviate or eliminate one or more of the drawbacks of the prior art.

The present invention concerns new hydrogel which show the following benefits:
Easier to inject,
More malleable,
can self-repair.

The invention also concerns the use of such gels, of particular interest to fill wrinkles and/or shape the face more accurately and with fewer traumas for the patient.

In one aspect of the invention, there is provided, glycosaminoglycans crosslinked by a first and a second linkage, wherein a) said first linkage comprises two ether bonds, one bond formed with a hydroxyl group of each a first and a second glycosaminoglycan; and b) said second linkage is via an alkoxyboronate ester anion formed between a boronate hemiester grafted to a first glycosaminoglycan and a diol function of a second glycosaminoglycan, wherein said diol function may be a backbone diol function or a diol portion of a diol functional moiety grafted to said second glycosaminoglycan.

In other words, glycosaminoglycans crosslinked by an irreversible linkage and a reversible linkage, wherein a) said irreversible linkage is via an irreversible linker which forms an ether bond with a backbone diol function of each of two glycosaminoglycans; and

b) said reversible linkage is via an alkoxyboronate ester anion formed between a boronate hemiester grafted to a first glycosaminoglycan and a diol function of a second glycosaminoglycan, wherein said diol function may be a backbone diol function or a diol function grafted to said second glycosaminoglycan.

The crosslinked glycosaminoglycans use a boronate hemiester to form a second linkage, although the linkages may be in any order. In one embodiment step b) is performed prior to step a). In one embodiment step a) is performed prior to step b). The crosslinked glycosaminoglycans according to the invention give a gel with improved properties (see example 11). In particular, the crosslinked glycosaminoglycans according to the invention give a more cohesive gel than a single crosslinked glycosaminoglycan, but also other improved rheological properties such as increased strength of doubly crosslinked networks due to boronate bonds when subjected to increasing stress.

Crosslinked glycosaminoglycans according to the invention further provide self-healing properties to the obtained gel (see e.g. FIG. 6, Example 12). The obtained gel is also easy to inject as the reversible bonds break when pushed through the syringe, and then quickly reform inside the body. The gels can be injected as preformed solids, because the solid gel can manage external damages and repair itself under a proper shear stress. Due to fast gelation kinetics after extrusion/injection, they recover their solid form almost immediately. Thus, before the gel reforms inside the body, the gel is malleable, until the reversible bonds reform. Thus, in one embodiment, the method provides a self-healing gel. The crosslinked glycosaminoglycans may optionally be further crosslinked.

The present disclosure provides new hydrogel products and related advantageous processes for preparing hydrogels made of crosslinked glycosaminoglycan (GAG) molecules having reversible linkages, and uses thereof. GAGs are negatively charged heteropolysaccharide chains which have a capacity to absorb large amounts of water. In the hydrogel products according to the disclosure, the crosslinked GAG molecule is the swellable polymer which provides the gel properties.

The polysaccharide according to the present disclosure is preferably a glycosaminoglycan (GAG). According to some embodiments, the glycosaminoglycan is selected from the group consisting of sulfated or non-sulfated glycosaminoglycans such as hyaluronan, chondroitin, chondroitin sulphate, heparan sulphate, heparosan, heparin, dermatan sulphate and keratan sulphate. According to some embodiments, the glycosaminoglycan is selected from the group consisting of hyaluronic acid, chondroitin and chondroitin sulfate, and mixtures thereof. According to some embodiments, the glycosaminoglycan is hyaluronic acid.

The synthesis of stable covalent ether bonds was carried out between hydroxyl groups of hyaluronic acid with a chemical crosslinking agent, preferably 1,4-butanediol diglycidyl ether (BDDE).

The reversible ester bonds were formed:
Between benzoboroxole modified hyaluronic acid (HA-BOR) and polyols modified hyaluronic acid (HA-polyols)
By benzoboroxole modified hyaluronic acid and the diol groups of hyaluronic acid.

As demonstrated in the appended examples, the crosslinked glycosaminoglycans according to the invention gives a gel with improved strength when subjected to increasing stress.

A diol function according to the invention may be any group comprising a diol, such as a 1,2-diol or a 1,3-diol, such as a sugar moiety, a sugar moiety derivative or a backbone diol function i.e. a diol which is part of the glycosaminoglycan chain. Suitable sugar derivatives are derivatives suitable for binding to a glycosaminoglycan. Such derivatives may be a thiol-modified mono- or disaccharide or an aminosugar. In certain embodiments of the invention, the diol portion is a vicinal diol. In other embodiments of the invention, the diol portion is not a vicinal diol.

As used herein, the term “backbone” refers to the polysaccharide chain in its native form i.e. groups grafted to the backbone are not part of the backbone. As an example, below the backbone of hyaluronic acid is shown.

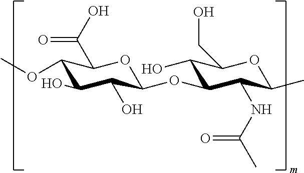

As used herein, the term “boronate hemiester” is to be interpreted as a compound of general formula BR(OR)(OH), as opposed to a boronic acid, which has a general formula BR(OH)₂, or a boronate ester which has a general formula BR(OR)₂. Each R, in this context, may independently represent any organic moiety since the purpose of these formulae relates to different boron functional groups.

A boronate ester is in equilibrium with its tetrahedral anionic form in water (below). The anionic form is an hydroxyboronate ester anion (Hall, D. G., 2011, Boronic Acids: Preparation and Applications in Organic Synthesis, Medicine and Materials, Second Edition, Wiley-VCH Verlag GmbH & Co.).

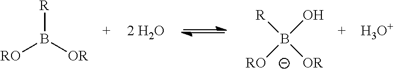

Thus, in general terms, an “alkoxyboronate ester anion” is to be understood as an anionic tetrahedral form, formed between a boronate ester and any alkoxy group, substituted or unsubstituted. An “alkoxyboronate ester anion” according to the invention, is an “alkoxyboronate ester anion” formed between a boronate hemiester and a backbone diol function of a glycosaminoglycan (below).

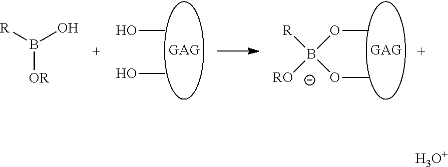

In one embodiment of this aspect of the invention the boronate hemiester is a compound comprising a 5-6-membered cyclic boronate hemiester moiety, sometimes referred to as a boroxole (Kotsubayashi et al. ACS Macro Lett. 2013, 2, 260-264). A five-membered boroxole is referred to as an oxaborole and a six-membered, an oxaborinine, see below. Thus, in one embodiment of this aspect of the invention the boronate hemiester is a compound comprising an oxaborole or an oxaborinine moiety.

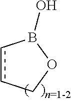

The present invention proposes new hydrogels:
in which the GAG chains are connected with reversible crosslinks in addition to covalent crosslinks (resulting for example from the reaction between GAG and a chemical crosslinking agent, preferably BDDE).

In one embodiment of this aspect of the invention the boronate hemiester is an optionally substituted benzoxaborole or benzoxaborinine. Benzoxaborol is sometimes referred to as benzoboroxole and the names may be used interchangeably (US 2014/0155305) The benzylic position of the boron atom in an optionally substituted benzoxaborole or benzoxaborinine stabilizes the empty p-orbital on the boron atom. Typically, the benzoxaborole or benzoxaborinine may be substituted with one or more of H, F, Cl, NO₂, C₁-C₃alkyl, C₁-C₃haloalkyl, C₁-C₃alkoxy, C₃-C₆cycloalkyl, phenyl, and a five- to six-membered heteroaromatic ring comprising 1 to 3 heteroatoms selected from O, N and S.

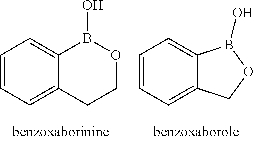

In one embodiment of this aspect of the invention, said second linkage is defined in Formula (I)

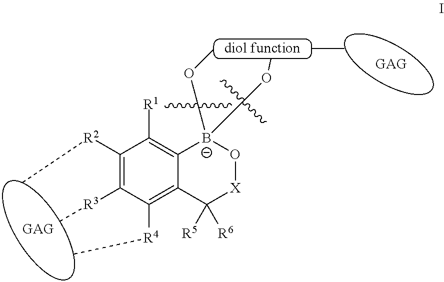

wherein
R¹ is selected from H, F, Cl, NO₂, C₁-C₃alkyl, C₁-C₃haloalkyl and C₁-C₃alkoxy;
R², R³ and R⁴ are independently selected from H, F, Cl, C₁-C₃haloalkyl, NO₂, C₁-C₃alkoxy, C₁-C₃alkyl and a linker, said linker binding covalently to said second glycosaminoglycan;
X is selected from CHR⁷ and a bond;
R⁵, R⁶ and R⁷ are independently selected from H, C₁-C₄alkyl, C₃-C₆cycloalkyl, phenyl, and a five- to six-membered heteroaromatic ring comprising 1 to 3 heteroatoms selected from O, N and S; and
wherein one of R², R³ and R⁴ is a linker.

A glycosaminoglycan, when grafted with a boronate hemiester, which is used according to the invention has a higher affinity to diol functions than phenylboronic acid of the prior art. This increased affinity is shown in example 3, where hyaluronic acid grafted with a boronate hemiester is shown to form a gel by crosslinking to a backbone diol function of another hyaluronic acid. The corresponding experiments using phenyl boronic acid failed to form gel. In addition, the gel formed when grafting a glycosaminoglycan with a boronate hemiester has self-healing properties, which is shown in example 4.

As used herein, the term “C₁-C₃haloalkyl” means both linear and branched chain saturated hydrocarbon groups, with 1 to 3 carbon atoms and with 1 to all hydrogens substituted by a halogen of different or same type. Examples of C₁-C₃haloalkyl groups include methyl substituted with 1 to 3 halogen atoms, ethyl substituted with 1 to 5 halogen atoms, and n-propyl or iso-propyl substituted with 1 to 7 halogen atoms.

As used herein, the term “C₁-C₃fluoroalkyl” means both linear and branched chain saturated hydrocarbon groups, with 1 to 3 carbon atoms and with 1 to all hydrogen atoms substituted by a fluorine atom. Examples of C₁-C₃fluoroalkyl groups include methyl substituted with 1 to 3 fluorine atoms, ethyl substituted with 1 to 5 fluorine atoms, and n-propyl or iso-propyl substituted with 1 to 7 fluorine atoms.

According to some embodiments, the glycosaminoglycan is selected from the group consisting of sulfated or non-sulfated glycosaminoglycans such as hyaluronan, chondroitin, chondroitin sulphate, heparan sulphate, heparosan, heparin, dermatan sulphate and keratan sulphate. According to some embodiments, the glycosaminoglycan is selected from the group consisting of hyaluronic acid, chondroitin and chondroitin sulfate, and mixtures thereof.

In one embodiment of this aspect of the invention, said glycosaminoglycans are hyaluronic acid. As demonstrated in the appended examples, the crosslinked glycosaminoglycans, said glycosaminoglycans being hyaluronic acid gives a gel with improved strength when subjected to increasing stress.

Hyaluronic acid (HA) is one of the most widely used biocompatible polymers for medical and cosmetic use. HA is a naturally occurring polysaccharide belonging to the group of glycosaminoglycans (GAGs). Hyaluronic acid consists of two alternating monosaccharides units, N-acetyl-D-glucosamine (GlcNAc) and D-glucuronic acid (GlcA), assembled by β(1→3) and β(1→4) glycosidic bonds, respectively. Hyaluronic acid and products derived from hyaluronic acid are widely used in the biomedical and cosmetic fields, for instance during viscosurgery and as a dermal filler.

Unless otherwise specified, the term “hyaluronic acid” encompasses all variants and combinations of variants of hyaluronic acid, hyaluronate or hyaluronan, of various chain lengths and charge states, as well as with various chemical modifications. That is, the term also encompasses the various hyaluronate salts of hyaluronic acid with various counter ions, such as sodium hyaluronate. The hyaluronic acid can be obtained from various sources of animal and non-animal origin. Sources of non-animal origin include yeast and preferably bacteria. The molecular weight of a single hyaluronic acid molecule is typically in the range of 0.1-10 kg/mol, but other molecular weights are possible. According to the invention, preferred molecular weights are in the range 50-3000 kg/mol, more preferably in the range 70-1000 kg/mol.

In one embodiment of this aspect of the invention, the molecular weight of the glycosaminoglycan is between 200-1500 kg/mol, preferably in the range 400-1100 kg/mol, more preferably 500-1000 kg/mol, more preferably 600-800 kg/mol. It has been experimentally observed that these ranges of molecular weights of the hyaluronic acid exhibit improved gel properties (e.g. G′ and G″), when grafted with a boronate hemiester.

The term “chondroitin” refers to GAGs having a disaccharide repeating unit consisting of alternating non-sulfated D-glucuronic acid and N-acetyl-D-galactosamine moieties. For avoidance of doubt, the term “chondroitin” does not encompass any form of chondroitin sulfate.

The term “chondroitin sulfate” refers to GAGs having a disaccharide repeating unit consisting of alternating D-glucuronic acid and N-acetyl-D-galactosamine moieties. The sulfate moiety can be present in various different positions. Preferred chondroitin sulfate molecules are chondroitin-4-sulfate and chondroitin-6-sulfate.

The chondroitin molecules can be obtained from various sources of animal and non-animal origin. Sources of non-animal origin include yeast and preferably bacteria. The molecular weight of a single chondroitin molecule is typically in the range of 1-500 kg/mol, but other molecular weights are possible.

The term “crosslinked glycosaminoglycans” or “crosslinked glycosaminoglycan molecules” refers herein to glycosaminoglycans comprising, typically covalent, crosslinks between the glycosaminoglycan molecule chains, which creates a continuous network of glycosaminoglycan molecules held together by the crosslinks.

The crosslinked GAG product is preferably biocompatible. This implies that no, or only very mild, immune response occurs in the treated individual. That is, no or only very mild undesirable local or systemic effects occur in the treated individual.

The crosslinked product according to the disclosure is a gel, or a hydrogel. That is, it can be regarded as a water-insoluble, but substantially dilute crosslinked system of GAG molecules when subjected to a liquid, typically an aqueous liquid.

Due to its significant liquid content, the gel product is structurally flexible and similar to natural tissue, which makes it very useful as a scaffold in tissue engineering and for tissue augmentation. It is also useful for treatment of soft tissue disorder and for corrective or aesthetic treatment. It is preferably used as an injectable formulation.

The hydrogel product may be present in an aqueous solution, but it may also be present in dried or precipitated form, e.g. in ethanol.

The hydrogel product is preferably injectable.

The hyaluronic acid can be obtained from various sources of animal and non-animal origin. Sources of non-animal origin include yeast and preferably bacteria. The molecular weight of a single hyaluronic acid molecule is typically in the range of 0.1-10 kg/mol, but other molecular weights are possible.

In certain embodiments the concentration of said hyaluronic acid is in the range of 1 to 100 mg/ml. In some embodiments the concentration of said hyaluronic acid is in the range of 2 to 50 mg/ml. In specific embodiments the concentration of said hyaluronic acid is in the range of 5 to 30 mg/ml or in the range of 10 to 30 mg/ml. In certain embodiments, the hyaluronic acid is permanently crosslinked (gel type B). Crosslinked hyaluronic acid comprises crosslinks between the hyaluronic acid chains, which creates a continuous network of hyaluronic acid molecules which is held together by reversible covalent crosslinks (gel type A and gel type C) or reversible covalent crosslinks in addition to permanent covalent crosslinks (gel type B).

Crosslinking of hyaluronic acid may be achieved by modification with a boroxole derivative and a polyol derivative to form linear HA-BOR and HA-polyol derivatives. The degree of substitution (DS) of these HA-conjugates can be varied in a range from 0.05 to 0.30 in order to tune the rheological behavior of the gels. Crosslinking of hyaluronic acid may be also achieved by modification with a chemical crosslinking agent and then, with a boroxole derivative and a polyol derivative. The chemical crosslinking agent may for example be selected from the group consisting of divinyl sulfone, multiepoxides and diepoxides. According to an embodiment, the hyaluronic acid is crosslinked by a bi- or polyfunctional crosslinking agent comprising two or more glycidyl ether functional groups. According to embodiments the chemical crosslinking agent is selected from the group consisting of 1,4-butanediol diglycidyl ether (BDDE), 1,2-ethanediol diglycidyl ether (EDDE) and diepoxyoctane. According to a preferred embodiment, the chemical crosslinking agent is 1,4-butanediol diglycidyl ether (BDDE).

A typical application of the resulting hydrogel product involves the preparation of injectable formulations for treatment of soft tissue disorders, including, but not limited to, corrective and aesthetic treatments.

In one embodiment of this aspect of the invention, said linker forms an amide bond or an ether bond with said first glycosaminoglycan;

Y is selected from a bond and C₁-C₆alkylene in which one or two CH₂ are optionally replaced by a group selected from O, NH and phenylene, said C₁-C₆alkylene being optionally substituted with 1 to 12 R⁸; and
R⁸ is selected from F, Cl, C₁-C₃alkyl, C₁-C₃haloalkyl, phenyl, OH, C₁-C₃hydroxyalkyl, C₁-C₃alkoxy, NH₂, N-C₁-C₃alkylamino, N,N-C₁-C₄dialkylamino. The grafting of boronate hemiester to said first glycosaminoglycan, said boronate hemiester being part of said second linkage according to Formula I, may be done for example via an ether bond by reacting for example a hydroxy group of the backbone of the glycosaminoglycan with an epoxy functionality of said linker. The grafting of boronate hemiester to said first glycosaminoglycan, said boronate hemiester being part of said second linkage according to Formula I, may also be done by using 4-(4,6-dimethoxy-1,3,5-triazin-2-yl)-4-methylmorpholinium chloride (DMTMM) to activate carboxylic groups on said second glycosaminoglycan and react the resulting species with an amine function of said linker to form a stable amide.

In one embodiment of this aspect of the invention, R² is a linker. When R² is used as the linker in the first linkage a gel was obtained.

In one embodiment of this aspect of the invention, said linker is H₂N—Y— or

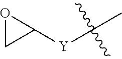

and forms an amide bond or an ether bond with said first glycosaminoglycan;
Y is selected from a bond and C₁-C₆alkylene in which one or two CH₂ are optionally replaced by a group selected from O, NH and phenylene, said C₁-C₆alkylene being optionally substituted with 1 to 12 R⁸; and
R⁸ is selected from F, Cl, C₁-C₃alkyl, C₁-C₃haloalkyl, phenyl, OH, C₁-C₃hydroxyalkyl, C₁-C₃alkoxy, NH₂, N-C₁-C₃alkylamino, N,N-C₁-C₄dialkylamino.

In one embodiment of this aspect of the invention, said linker is —NR⁹—Y— and forms an amide bond with said second glycosaminoglycans, wherein R⁹ is selected from hydrogen, C₁-C₃alkyl and C₁-C₃fluoroalkyl; and

Y is a bond or an unsubstituted C₁-C₆alkylene. The grafting of the boronate hemiester to said first glycosaminoglycan, said boronate hemiester being part of said second linkage according to Formula I, may be done by using 4-(4,6-dimethoxy-1,3,5-triazin-2-yl)-4-methylmorpholinium chloride (DMTMM) to activate carboxylic groups on said first glycosaminoglycan and react the resulting species with an amine function (HR⁹N—Y—) of said linker to form a stable amide.

In one embodiment of this aspect of the invention, R⁹ is hydrogen.

In one embodiment of this aspect of the invention, the boronate hemiester is

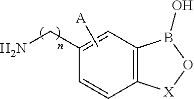

wherein A is selected from H, F, CF₃, NO₂, OCH₃ and CH₃;
n is selected from 0, 1, 2 and 3; and
X is selected from CH₂, CH₂—CH₂, CH—NC₅H₁₁ (CH-piperidine) and C(CH₃)₂.

In one embodiment of this aspect of the invention,

R¹, R³ and R⁴ are independently selected from H, F, OCH₃, CF₃ and CH₃;
R² is a linker;
said linker is —HN—Y— and forms an amide bond with said first glycosaminoglycan;
Y is a bond or an unsubstituted C₁-C₃alkylene;
X is a bond or CH₂; and
R⁵ and R⁶ are independently selected from H and C₁-C₃alkyl.

In one embodiment of this aspect of the invention, said boronate hemiester is selected from

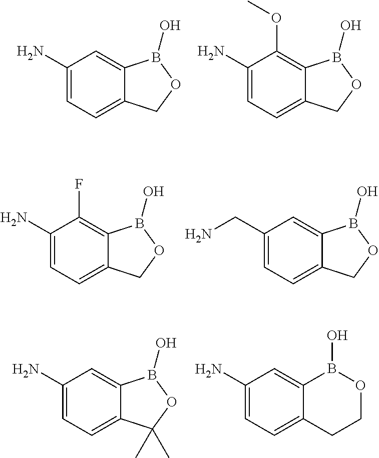

wherein the boronate hemiester is grafted to said first glycosaminoglycan by that the —NH₂ group of the boronate hemiester forms an amide with a backbone carboxylate group of said first glycosaminoglycan.

In one embodiment of this aspect of the invention, said second linkage having a structure of Formula (II)

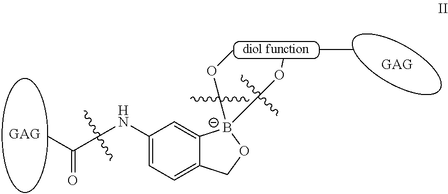

In one embodiment of this aspect of the invention, said diol function is a backbone diol function.

In one embodiment of this aspect of the invention, said diol function is a diol portion of a diol functional moiety grafted to said second glycosaminoglycan.

In one embodiment of this aspect of the invention, said diol portion is selected from a monosaccharide, a disaccharide and an alditol or a derivative thereof.

In one embodiment of this aspect of the invention, said diol portion is selected from a hexose, a dihexose and a C₆alditol or a derivative thereof.

In one embodiment of this aspect of the invention, said diol portion is selected from maltose, fructose, lactose and sorbitol or a derivative thereof. Suitable derivatives for are maltose, fructose, lactose and sorbitol derivatives suitable for binding to a glycosaminoglycan. Such derivatives may be a mono- or di-saccharide-disulfide or an aminosugar.

In one embodiment of the invention said diol portion is selected from Maltose-disulfide, Lactobionic-disulfide, 1-amino-1-deoxy-D-fructose and 1-amino-1-deoxy-D-sorbitol.

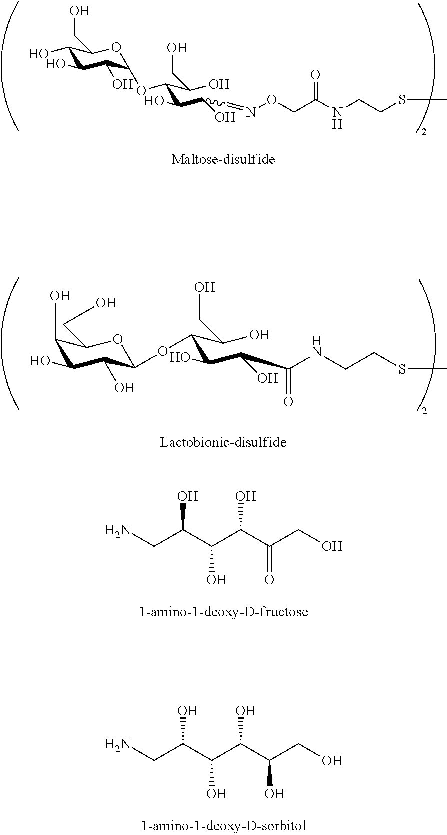

In one embodiment of this aspect of the invention, said diol portion is a ketose or a derivative thereof.

In one embodiment of this aspect of the invention, said diol portion is selected from maltose, fructose, lactose and sorbitol or an amino- or a derivative thereof.

In one embodiment of this aspect of the invention, said diol portion is fructose or a derivative thereof.

In one embodiment of this aspect of the invention, said first linkage is a 1,4-butanediol di-(propan-2,3-diolyl)ether linkage.

In one aspect of the invention, there is provided a method of crosslinking glycosaminoglycans, comprising the steps of:
forming a linkage comprising two ether bonds, one bond formed with a hydroxyl group of each a first and a second glycosaminiglycan;
grafting said second glycosaminoglycan with a boronate hemiester and crosslinking said first glycosaminoglycan with said second glycosaminoglycan by forming an alkoxyboronate ester anion linkage between the boronate hemiester of said first glycosaminoglycan and a diol function of said second glycosaminoglycan, wherein said diol function may be a backbone diol function or a diol portion of a diol functional moiety grafted to said second glycosaminoglycan.

The method according to the invention further provide self-healing properties to the obtained gel (see e.g. FIG. 6, Example 12). A gel produced by the method according to the invention is also easy to inject as the reversible bonds break when pushed through the syringe, and then quickly reform inside the body. The gels can be injected as preformed solids, because the solid gel can manage external damages and repair itself under a proper shear stress. Due to fast gelation kinetics after extrusion/injection, they recover their solid form almost immediately. Thus, before the gel reforms inside the body, the gel is malleable, until the reversible bonds reform. Thus, in one embodiment, the method provides a self-healing gel.

In one embodiment, the boronate hemiester has higher affinity towards diols, such as sugars or derivatives thereof, than for example phenylboronic acid.

Different embodiments of the method according to the invention may be employed to synthesize doubly crosslinked hyaluronic acid gels gels: i) crosslinking of a mixture of hyaluronic acid grafted with a boronate hemiester derivative and of a hyaluronic acid optionally grafted with a diolfunctional moiety by reaction of HA hydroxyl groups with BDDE; ii) grafting of BOR or fructose moieties on HA-BDPE gel particles by a peptide-like coupling reaction. In other words, in method ii), the crosslinked GAG gels can present the form of gel particles. The gel particles have an average size in the range of 0.01-5 mm, preferably 0.1-0.8 mm, such as 0.2-0.5 mm or 0.5-0.8 mm. They are covalently crosslinked by the reaction between GAG and BDDE and further modified with a boroxole derivative and/or a polyol derivative. Therefore, the hydrogel product can consist of gel particles which are connected together via reversible bonds formed by the reaction between the boroxole moieties and GAG units or polyol groups grafted on the GAG.

The hydrogel product may also comprise a portion of linear GAG modified with polyol groups or boroxole moieties and gel particles covalently crosslinked with BDDE and modified with a boroxole derivative and/or a polyol derivative.

In one embodiment of this aspect of the invention, said boronate hemiester is a compound of Formula (III),

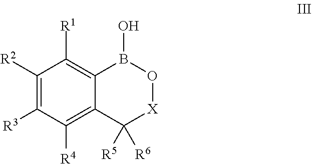

wherein
R¹ is selected from H, F, Cl, NO₂, C₁-C₃alkyl, C₁-C₃haloalkyl and C₁-C₃alkoxy;
R², R³ and R⁴ are independently selected from H, F, Cl, C₁-C₃haloalkyl, NO₂, C₁-C₃alkoxy, C₁-C₃alkyl and a linker binding covalently to said second glycosaminoglycan;
X is selected from CHR³ and a bond; and
R⁵, R⁶ and R⁷ are independently selected from H, C₁-C₄alkyl, C₃-C₆cycloalkyl, phenyl, and a five- to six-membered heteroaromatic ring comprising 1 to 3 heteroatoms selected from O, N and S, wherein one of R², R³ and R⁴ is a linker.

In one embodiment of this aspect of the invention, said first and said second glycosaminoglycans are hyaluronic acid.

In one embodiment of this aspect of the invention,

said linker forms an amide bond or an ether bond to said second glycosaminoglycan;
Y is selected from a bond and C₁-C₆alkylene in which one or two CH₂ are optionally replaced by a group selected from O, NH and phenylene, said C₁-C₆alkylene being optionally substituted with 1 to 12 R⁸; and
R⁸ is selected from F, Cl, C₁-C₃alkyl, C₁-C₃haloalkyl, phenyl, OH, C₁-C₃hydroxyalkyl, C₁-C₃alkoxy, NH₂, N-C₁-C₃alkylamino, N,N-C₁-C₄dialkylamino.

In one embodiment of this aspect of the invention, R² is a linker.

In one embodiment of this aspect of the invention, said linker is HR⁹N—Y— and forms an amide bond with said second glycosaminoglycan, wherein R⁹ is selected from hydrogen, C₁-C₃alkyl and C₁-C₃fluoroalkyl; and

Y is a bond or an unsubstituted C₁-C₆alkylene.

In one embodiment of this aspect of the invention, R¹, R³ and R⁴ are independently selected from H, F, OCH₃, CF₃ and CH₃;

R² is a linker;
said linker is H₂N—Y— and forms an amide bond with said second glycosaminoglycan;
Y is a bond or an unsubstituted C₁-C₃alkylene;
X is a bond or CH₂; and
R⁵ and R⁶ are independently selected from H and C₁-C₃alkyl.

In one embodiment of this aspect of the invention, said boronate hemiester is selected from

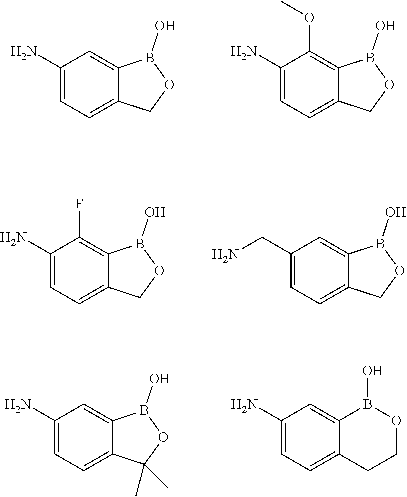

wherein the boronate hemiester is grafted to said second glycosaminoglycan by that the —NH₂ group of the boronate hemiester forms an amide with a backbone carboxylate group of said second glycosaminoglycan.

In one embodiment of this aspect of the invention, said boronate hemiester being

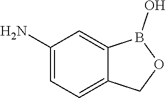 [SMILES: Nc1ccc2c(c1)B(O)OC2]

In one embodiment of this aspect of the invention, said diol function is a backbone diol function. A boronate hemiester has higher affinity towards diols than for example phenylboronic acid. Thus, to make a gel, it is not necessary to graft a sugar derivative on said second glycosaminoglycan.

In some embodiments, a glycosaminoglycan can be grafted to a higher degree of substitution with a boronate hemiester than a corresponding glycosaminoglycan grafted with a phenylboronic acid. This may be useful when forming a gel together with a glycosaminoglycan grafted with a diol-functional moiety, particularly a self-healing gel.

In one embodiment of this aspect of the invention, said diol function is a diol portion of a diol functional moiety grafted to said second glycosaminoglycan.

In one embodiment of this aspect of the invention, said diol portion is selected from a monosaccharide, a disaccharide and an alditol or a derivative thereof.

In one embodiment of this aspect of the invention, said diol portion is selected from a hexose, a dihexose and a C₆alditol or a derivative thereof.

In one embodiment of this aspect of the invention, said diol portion is selected from maltose, fructose, lactose and sorbitol or a derivative thereof.

In one embodiment of this aspect of the invention, said diol portion is fructose or a derivative thereof.

In one embodiment of this aspect of the invention, the step of forming the linkage comprising two ether bonds is performed prior to the step of grafting said second glycosaminoglycan with a boronate hemiester.

In one embodiment of this aspect of the invention, the linkage comprising two ether bonds is a 1,4-butanediol di-(propan-2,3-diolyl)ether linkage.

In one aspect of the invention there is provided use of a boronate hemiester in the manufacture of glycosaminoglycans crosslinked by a first and a second linkage, wherein said first linkage comprises two ether bonds, one bond formed with a hydroxyl group of each a first and a second glycosaminoglycan; and

said second linkage is via an alkoxyboronate ester anion formed between a diol function of said second glycosaminoglycan and a boronate hemiester grafted to said second glycosaminoglycan, wherein said diol function may be a backbone diol function or a diol portion of a diol functional moiety grafted to said second glycosaminoglycan.

The use of a boronate hemiester in the manufacture of crosslinked glycosaminoglycans according to the invention further provide self-healing properties to the obtained gel (see e.g. FIG. 6, Example 12). The obtained gel is also easy to inject as the reversible bonds break when pushed through the syringe, and then quickly reform inside the body. The gels can be injected as preformed solids, because the solid gel can manage external damages and repair itself under a proper shear stress. Due to fast gelation kinetics after extrusion/injection, they recover their solid form almost immediately. Thus, before the gel reforms inside the body, the gel is malleable, until the reversible bonds reform. Thus, in one embodiment, the method provides a self-healing gel. The crosslinked glycosaminoglycans may optionally be further crosslinked.

In one embodiment of this aspect of the invention, said boronate hemiester is a compound of Formula (IV)

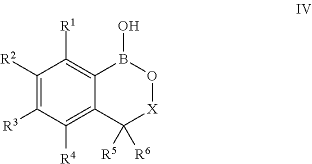

wherein
R¹ is selected from H, F, Cl, NO₂, C₁-C₃alkyl, C₁-C₃haloalkyl and C₁-C₃alkoxy;
R², R³ and R⁴ are independently selected from H, F, Cl, C₁-C₃haloalkyl, NO₂, C₁-C₃alkoxy, C₁-C₃alkyl and a linker capable of binding covalently to said second glycosaminoglycan;
X is selected from CHR⁷ and a bond; and
R⁵, R⁶ and R⁷ are independently selected from H, C₁-C₄alkyl, C₃-C₆cycloalkyl, phenyl, and a five- to six-membered heteroaromatic ring comprising 1 to 3 heteroatoms selected from O, N and S,
wherein one of R², R³ and R⁴ is a linker.

In one embodiment of this aspect of the invention, said glycosaminoglycans are hyaluronic acid.

In one embodiment of this aspect of the invention, said linker is capable of forming an amide bond or an ether bond to said second glycosaminoglycan;

Y is selected from a bond and C₁-C₆alkylene in which one or two CH₂ are optionally replaced by a group selected from O, NH and phenylene, said C₁-C₆alkylene being optionally substituted with 1 to 12 R⁸; and
R⁸ is selected from F, Cl, C₁-C₃alkyl, C₁-C₃haloalkyl, phenyl, OH, C₁-C₃hydroxyalkyl, C₁-C₃alkoxy, NH₂, N-C₁-C₃alkylamino, N,N-C₁-C₄dialkylamino.

In one embodiment of this aspect of the invention, R² is a linker.

In one embodiment of this aspect of the invention, said linker is HR⁹N—Y— and forms an amide bond with said second glycosaminoglycan, wherein R⁹ is selected from hydrogen, C₁-C₃alkyl and C₁-C₃fluoroalkyl; and

Y is a bond or an unsubstituted C₁-C₆alkylene.

In one embodiment of this aspect of the invention R¹, R³ and R⁴ are independently selected from H, F, CF₃ and CH₃;

R² is a linker;
said linker is H₂N—Y— and capable of forming an amide bond with said second glycosaminoglycan;
Y is a bond or an unsubstituted C₁-C₃alkylene;
X is a bond or CH₂; and
R⁵ and R⁶ are independently selected from H and C₁-C₃alkyl.

In one embodiment of this aspect of the invention, said boronate hemiester is selected from

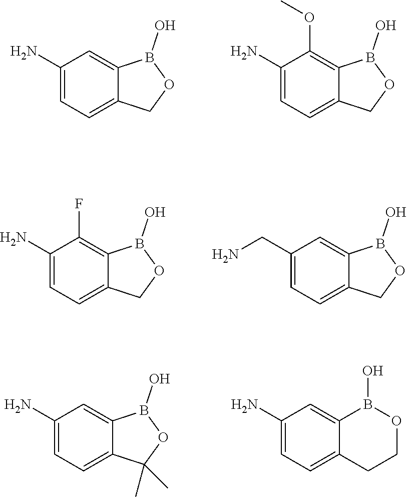

wherein the boronate hemiester is grafted to said second glycosaminoglycan by that the —NH₂ group of the boronate hemiester forms an amide with a backbone carboxylate group of said second glycosaminoglycan.

In one embodiment of this aspect of the invention, said boronate hemiester being

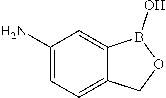 [SMILES: Nc1ccc2c(c1)B(O)OC2]

In one embodiment of this aspect of the invention, said diol portion is selected from a monosaccharide, a disaccharide and an alditol or a derivative thereof.

In one embodiment of this aspect of the invention, said diol portion is selected from a hexose, a dihexose and a C₆alditol or a derivative thereof.

In one embodiment of this aspect of the invention, said diol portion is selected from maltose, fructose, lactose and sorbitol or a derivative thereof.

In one embodiment of this aspect of the invention, said diol portion is fructose or a derivative thereof.

In one aspect of the invention there is provided a polymer composition comprising crosslinked glycosaminoglycans according to the invention and an aqueous buffer.

In one embodiment of this aspect of the invention, said crosslinked glycosaminoglycans are produced according to the method of the invention.

Water-absorbing gels, or hydrogels, are widely used in the biomedical field. They are generally prepared by chemical crosslinking of polymers to infinite networks. While native hyaluronic acid and certain crosslinked hyaluronic acid products absorb water until they are completely dissolved, crosslinked hyaluronic acid gels typically absorb a certain amount of water until they are saturated, i.e. they have a finite liquid retention capacity, or swelling degree.

According to related aspects, the present disclosure also provides use of the hydrogel product as a medicament, such as in the treatment of soft tissue disorders. There is provided a method of treating a patient suffering from a soft tissue disorder by administering to the patient a therapeutically effective amount of the hydrogel product. There is also provided a method of providing corrective or aesthetic treatment to a patient by administering to the patient a therapeutically effective amount of the hydrogel product.

According to other aspects illustrated herein, there is provided a hydrogel product obtained by the inventive method for use as a medicament.

According to other aspects illustrated herein, there is provided a hydrogel product obtained by the inventive method for use in the treatment of soft tissue disorders.

According to other aspects illustrated herein, there is provided the use of a hydrogel product obtained by the inventive method for the manufacture of a medicament for treatment of soft tissue disorders.

According to other aspects illustrated herein, there is provided a method of treating a patient suffering from a soft tissue disorder by administering to the patient a therapeutically effective amount of a hydrogel product obtained by the inventive method.

According to other aspects illustrated herein, there is provided a method of providing corrective or aesthetic treatment to a patient by administering to the patient a therapeutically effective amount of a hydrogel product obtained by the inventive method.

According to other aspects illustrated herein, there is provided a method of cosmetically treating skin, which comprises administering to the skin a hydrogel product obtained by the inventive method.

Other aspects and preferred embodiments of the present invention will be evident from the appended examples.

The term “molecular weight” as used herein in connection with various polymers, e.g. polysaccharides, refers to the weight average molecular weight, M_{W}, of the polymers, which is well defined in the scientific literature. The weight average molecular weight can be determined by, e.g., static light scattering, small angle neutron scattering, X-ray scattering, and sedimentation velocity. The unit of the molecular weight for polymers is g/mol. The person skilled in the art realizes that the present invention by no means is limited to the preferred embodiments described herein. On the contrary, many modifications and variations are possible within the scope of the appended claims. Additionally, variations to the disclosed embodiments can be understood and effected by the skilled person in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage.

In one aspect of the invention, there is provided novel hydrogels synthesized by reversible crosslinking of boronate ester bonds based on benzoboroxole modified hyaluronic acid (HA-BOR).

In one aspect of the invention, there is provided a polymer composition comprising glycosaminoglycans (GAG) crosslinked by reversible boronate ester bonds.

In one embodiment of this aspect of the invention said GAG is hyaluronic acid (HA).

In one embodiment of this aspect of the invention, said boroxole (BOR) modified hyaluronic acid (HA) polymer grafted at the carboxylate group comprising boroxole.

In one embodiment of this aspect of the invention, the polymer composition comprises a mixture of:

a) polymer grafted at the carboxylate group comprising boroxole; and

b) polyol modified hyaluronic acid (HA) polymer grafted on at least a hydroxyl with a group comprising a polyol.

In one embodiment of this aspect of the invention, the polymer composition comprises hyaluronic acid, wherein the polymer comprises doubly crosslinking based on biopolymer combining covalent ether bonds and reversible ester bonds, wherein, the stable covalent ether bonds is carried out between hydroxyl group of hyaluronic acid with 1,4-butanediol diglycidyl ether (BDDE), and wherein the reversible ester bonds are formed between benzoboroxole modified hyaluronic acid and polyols modified hyaluronic acid.

According to one embodiment of this aspect of the invention, the derivative of the benzoboroxole is

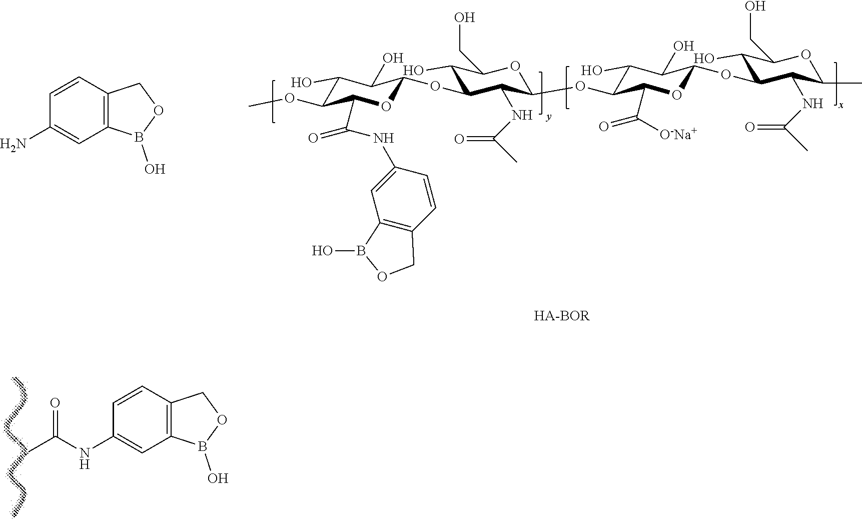

Boronate ester bonds are formed between benzoboroxole and diol groups on HA chain. The product obtained can be represented as below (formula IV). Gels behavior has been demonstrated by rheological analysis.

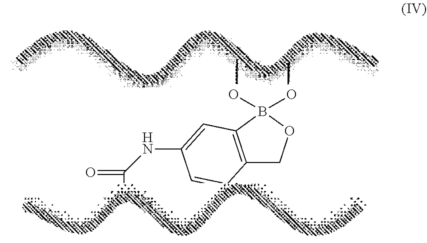

In one aspect of the invention, there is provided a polymer composition comprising a mixture of:

a) Boroxole modified HA polymer grafted on at the carboxylate group with a group comprising boroxole, and

b) Polyol, preferably monosaccharide, disaccharide and diol modified HA, and more preferably mono-, disaccharide and cis-diol modified HA polymer grafted on at least a hydroxyl group or at the carboxylate group.

More specifically, polyols that can be used to form derivatives with HA are preferably fructose, maltose, glucose, lactose, mannose, galactose, sorbitol, or glycerol.

HA-BOR:

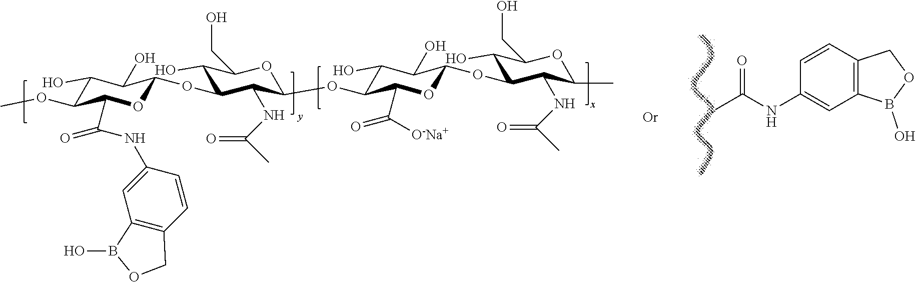

In one embodiment, the polyols consist preferably of: Maltose, Lactose, Fructose and Sorbitol.

Maltose:

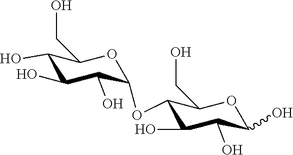

Fructose:

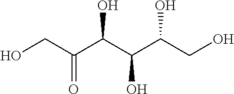 [SMILES: O=C(CO)[C@@H](O)[C@H](O)[C@H](O)CO]

Sorbitol:

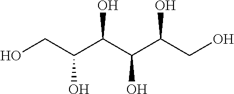 [SMILES: OC[C@@H](O)[C@@H](O)[C@H](O)[C@@H](O)CO]

Lactose:

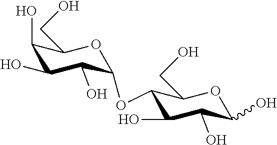

HA-polyols obtained are, as examples:

HA-maltose:

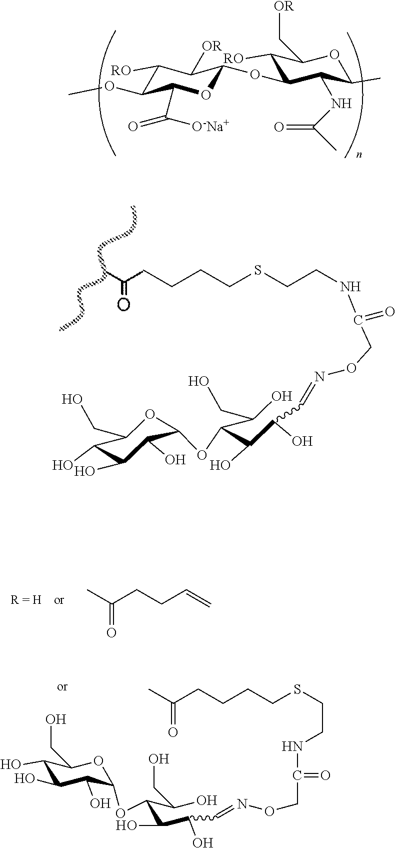

HA-fructose:

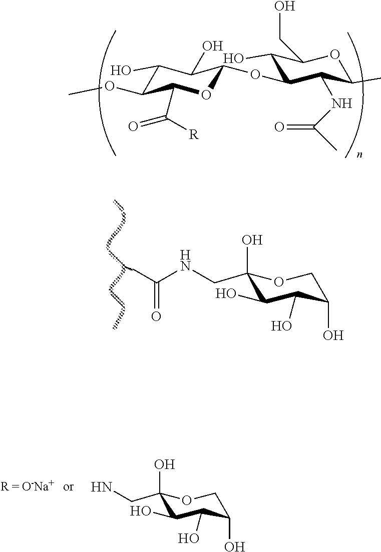

HA-sorbitol:

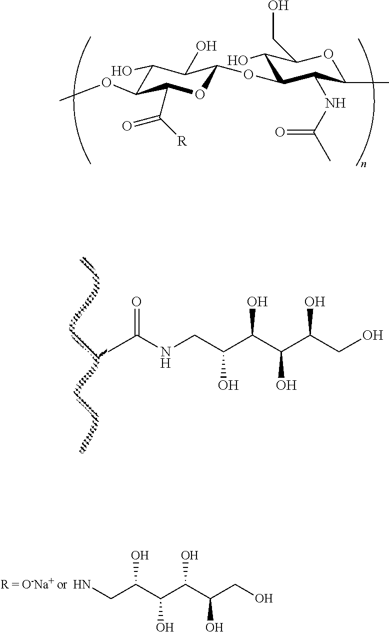

HA-lactobionic:

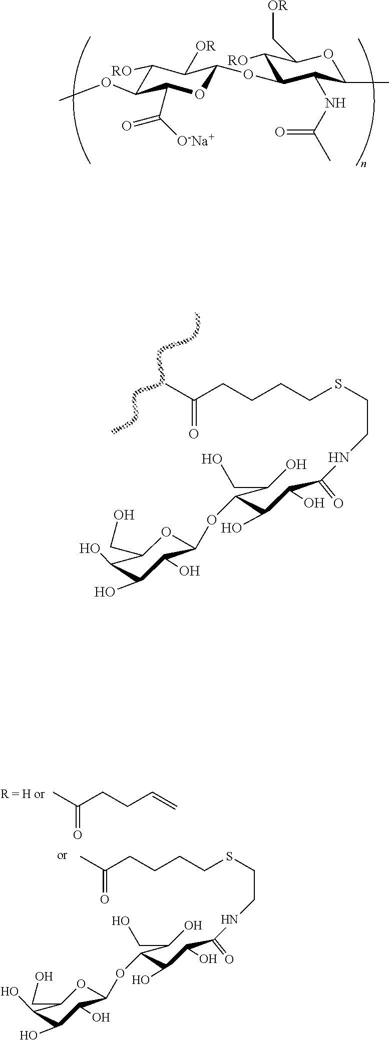

The hydrogel combining HA-BOR and HA-polyols obtained according to the invention is, for example:

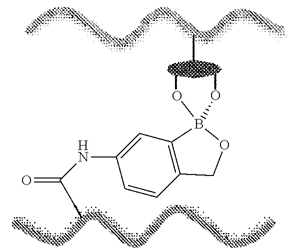

In the text of the present application, this symbol [symbol] represents the polyol.

### BRIEF DESCRIPTION OF THE DRAWINGS

FIG. 1: Gel obtained with HA-BOR.

FIG. 2: Rheological analysis: measurement of G′ and G″ for HA-BOR with HA M_{W} 600 kg/mol (HA600), [PS] of 15 g/L (HA-BOR derivative solubilized in ultrapure water at 30 g/L, followed by addition of 0.02 M HEPES buffer containing 0.3 M NaCl pH 7.4).

FIG. 3: Self-healing behavior of a HA-BOR hydrogel: application of gradually increasing stress values from 1800 to 2100 Pa for 2 min, intercalated with periods of application of a strain fixed at 5% for 3 min (frequency fixed at 1 Hz).

FIG. 4: Rheological analysis of HA-BOR/HA-fructose mixtures in 0.01M HEPES buffer containing 0.15M NaCl at different pH (from 4 to 8).

FIG. 5: Schematic structure of double crosslinked glycosaminoglycans.

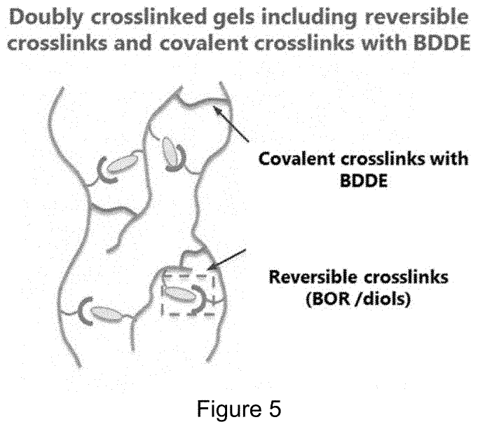

FIG. 6: Recovery of G′ and G″ as a function of time post-extrusion of a HA-DMABOR gel (M_{W}=600 kg/mol) through a 27 gauge needle.

### EXAMPLES

The following terms and characteristics will be used in the examples and results shown. The definitions are the one hereafter:

Mw—Molecular Weight: The mass average molecular mass

DS—Degree of Substitution The term “degree of substitution” (DS) as used herein in connection with various polymers, e.g. polysaccharides, refers to the average number of substituting group per repeating disaccharide unit

[PS]—The polysaccharide concentration (g/L)

G′: storage (elastic) modulus (in Pa)

G″: loss (viscous) modulus (in Pa)

G′ 1 Hz: storage modulus (in Pa) measured at a frequency of 1 Hz

G″ 1 Hz: loss modulus (in Pa) measured at a frequency of 1 Hz

Gel-like behavior: G′>G″ within the whole range of frequency covered (0.01-10 Hz)

Viscoelastic behavior: viscous (G′<G″) and elastic (G′>G″) behavior observed within the range of frequency covered (0.01-10 Hz).

ABOR: 5-Amino-2-methylphenylboronic acid

AMBOR: 6-(Aminomethyl)benzo[c][1,2]oxaborol-1(3H)-ol

APBA: 3-Aminophenylboronic acid

BDDE: 1,4-Butanediol diglycidyl ether

BDPE: 1,4-butanediol di-(propan-2,3-diolyl)ether

DMABOR: 6-Amino-3,3-dimethylbenzo[c][1,2]oxaborol-1(3H)-ol

DMF: Dimethylformamide

DMTMM: 4-(4,6-Dimethoxy-1,3,5-triazin-2-yl)-4-methylmorpholinium chloride

HEPES: 4-(2-Hydroxyethyl)-1-piperazineethanesulfonic acid

PBS: Phosphate buffered saline

TNBS: 2,4,6-Trinitrobenzenesulfonic acid

Without desiring to be limited thereto, the present invention will in the following be illustrated by way of examples.

### Example 1: Synthesis of HA-BOR

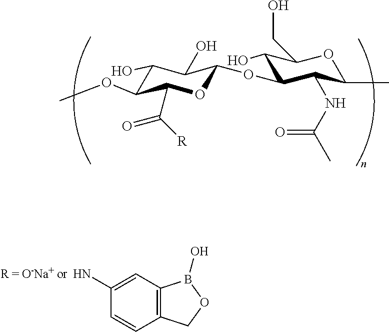

The amine-acid coupling agent 4-(4,6-dimethoxy-1,3,5-triazin-2-yl)-4-methylmorpholinium chloride (DMTMM) was dissolved in 1 mL of water and was added to a solution of native HA in a mixture of water/DMF (3/2, v/v). A concentration of HA in the reaction medium of 3 g/L was used for HA samples of 75 and 100 kg/mol, whereas 2 g/L was used for HA with 600 kg/mol. Then, 5-amino-2-hydroxymethylphenylboronic acid hydrochloride (1-hydroxy-3H-2,1-benzoxaborol-amine, ABOR) solubilized in 1 mL of water was added to the reaction medium. The pH was adjusted to 6.5 using 0.5 M HCl or NaOH and the reaction was kept under stirring at room temperature for 24 h. The product was purified by diafiltration with ultrapure water and was recovered by freeze-drying. The degree of substitution (DS) of HA-BOR was determined by ¹H NMR (DS_{NMR}), and were also estimated from the reaction kinetics performed using 2,4,6-Trinitrobenzene Sulfonic Acid (DS_{TNBS}). This method consisted in quantifying the free primary amines in the reaction medium as a function of time. Table 1 summarizes the DMTMM/HA and BOR/HA molar ratios used for the syntheses with different M_{W} HA, as well as the DS and the yields of HA-BOR conjugates.

HA-BOR: ¹H NMR (400 MHz, D₂O) δ_{H} (ppm) 4.55 (H-1 from N-acetylglucosamine unit), 4.25 (H-1 from glucuronic acid), 3.9-3.1 (H-2, H-3, H-4, H-5, H-6 protons of HA), 2.08 (CH₃—CO from HA), 7.95 (s, 1H, NH—C—CH—C—B from Ph), 7.72 (m, 1H, C—CH—CH—C—C—B from Ph), 7.55 (m, 1H, C—CH—CH—C—C—B from Ph), 5.13 (s, 2H, CH₂—O—B).

### Example 2 : Synthesis of HA-PBA (Comparative Example)

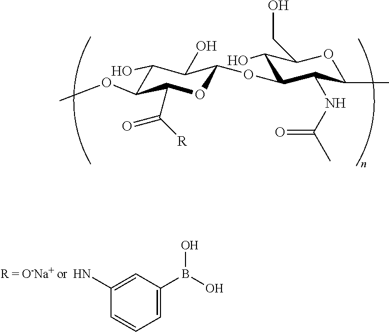

Grafting of phenylboronic acid was done according to Example 1, but using 3-aminophenylboronic acid hemisulfate salt (APBA) instead of 5-amino-2-hydroxymethylphenylboronic acid hydrochloride (ABOR). The degree of substitution (DS) of HA-PBA was determined by ¹H NMR (DS_{NMR}), and were also estimated from the reaction kinetics performed using 2,4,6-Trinitrobenzene Sulfonic Acid (DS_{TNBS}). This method consisted in quantifying the free primary amines in the reaction medium as a function of time. Table 1 summarizes the DMTMM/HA and PBA/HA molar ratios used for the syntheses with different M_{W} HA, as well as the DS and the yields of HA-PBA conjugates.

HA-PBA: ¹H NMR (400 MHz, D₂O) δ_{H} (ppm) 4.55 (H-1 from N-acetylglucosamine unit), 4.25 (H-1 from glucuronic acid), 3.9-3.1 (H-2, H-3, H-4, H-5, H-6 protons of HA), 2.08 (CH₃—CO from HA), 7.93 (s, 1H, NH—C—CH—C—B from Ph), 7.7 (m, 2H, C—CH—CH—CH—C—B from Ph), 7.55 (m, 1H, C—CH—CH—CH—C—B from Ph).

TABLE 1
Syntheses of HA-BOR and HA-PBA.

| HA-boronic | M_{w} HA | DMTMM/HA | BOR or PBA/HA | | | Yield |
|---|---|---|---|---|---|---|
| acid derivative | (Kg/mol) | molar ratio | molar ratio | DS_{NMR}^{a} | DS_{TNBS} | (%)^{b} |
| HA-BOR | 75 | 1 | 0.16 | 0.16 | 0.16 | 75 |
| HA-BOR | 100 | 1 | 0.16 | 0.12 | 0.14 | 85 |
| HA-BOR | 600 | 1 | 0.14 | 0.11 | 0.13 | 75 |
| HA-PBA | 75 | 1 | 0.16 | 0.16 | 0.16 | 75 |
| HA-PBA | 100 | 1 | 0.16 | 0.16 | 0.16 | 77 |
| HA-PBA | 600 | 1 | 0.14 | 0.14 | 0.14 | 78 |

^{a}DS by ¹H NMR: 10% of accuracy.
^{b}HA-BOR or HA-PBA yield: calculation considering the DS_{NMR}.

### Example 3: Synthesis of HA-BOR Gels

HA-BOR gels were prepared by solubilizing the HA-BOR derivative in 0.01 M HEPES buffer with 0.15 M NaCl at physiological pH. The characteristics of the obtained gels are shown in Table 2.

TABLE 2
Characteristics of HA-BOR hydrooel ([PS] = 15 g/L).

| | DS | | G′ | G″ | |
|---|---|---|---|---|---|
| HA-boronic acid | HA-boronic | Mw HA | 1 Hz | 1 Hz | Rheological |
| derivative | acid derivative | (kg/mol) | (Pa) | (Pa) | behavior |
| HA-benzoboroxole | 0.1 | 600 | 470 | 145 | Gel |
| HA-benzoboroxole | 0.1 | 1000 | 56 | 36 | Viscoelastic |
| Native HA | — | 600 | 2 | 8 | Viscous |
| Native HA | — | 1000 | 27 | 33 | Viscoelastic |

Boronate ester bonds are formed between benzoboroxole and diol groups HA. Gels behavior has been demonstrated by rheological analysis.

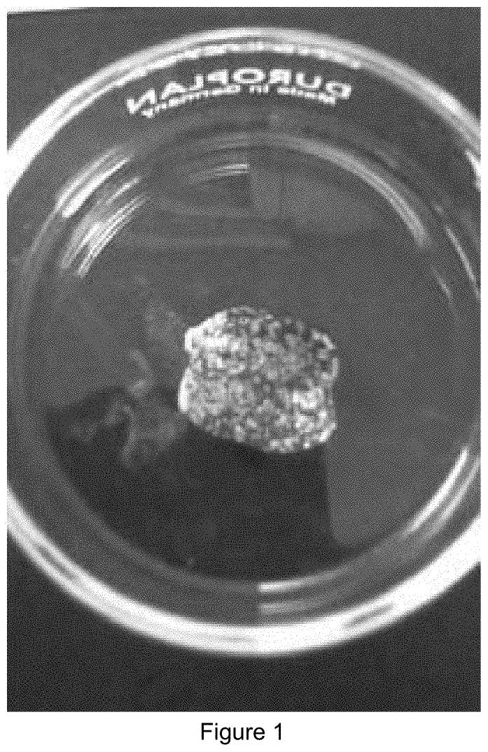

Surprinsingly, when coupling HA chains with benzoboroxole only, obtained hydrogels present good gel behaviour (FIG. 1).

### Example 4: Comparison of HA-BOR Gel to HA-PBA Gel and Native HA Gel

HA-BOR gel preparation:

HA-1-hydroxy-3H-2,1-benzoxaborol-amine (HA-BOR derivative) was solubilized in ultrapure water (pH 5-6) at 30 g/L for 24 h under continuous stirring at 4° C., followed by addition of 0.02M HEPES buffer containing 0.3M NaCl pH 7.4.

HA-PBA and native HA samples preparation:

HA-PBA or native HA was solubilized in ultrapure water (pH 5-6) at 30 g/L for 24 h under continuous stirring at 4° C., followed by addition of 0.02M HEPES buffer containing 0.3M NaCl pH 7.4. The solutions were stirred during 8 h at 4° C.

Results:

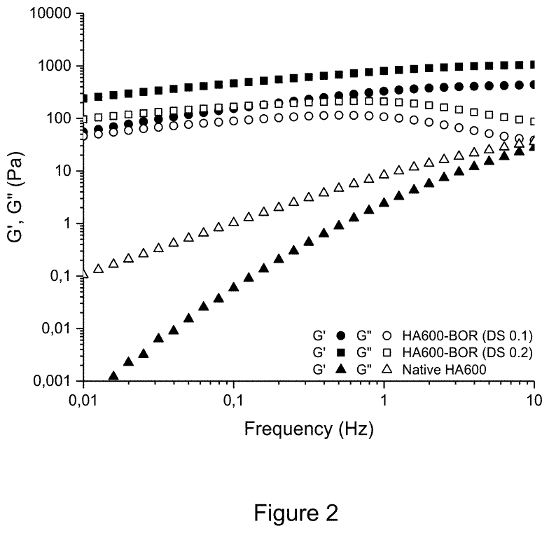

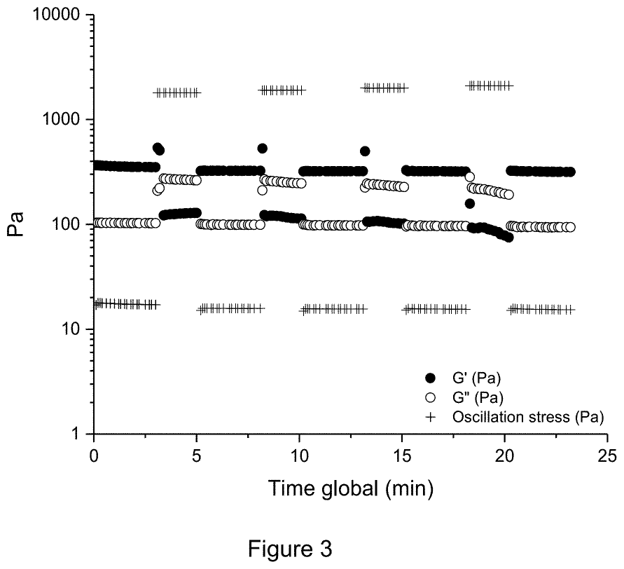

Within 8 h of stirring at 4° C., a final gel was obtained with a polymer concentration of 15 g/L and pH 7. Gels prepared using HA-BOR with M_{W} of 1000 kg/mol may require a longer time of solubilization (24 to 48 h). Characteristics of the resulting gels or viscous mixtures are shown in Table 3 and in FIG. 2. Self-healing properties of a dynamic gel of HA-BOR (C_{HA}=15 g/L) at 25° C. were investigated by, while measuring G′ and G″, applying successive stress values from 1800 to 2100 Pa for 2 min. These were intercalated with short time periods in which low stress values (corresponding to 5% strain) were applied for 3 min. This experiment demonstrated the stress recovery of the HA-BOR gel after 4 cycles of stress-induced breakdowns. Large stress (from 1800 to 2100 Pa) inverted the values of G′ (filled circles) and G″ (empty circles), indicating breakage of crosslinks and conversion to solution state. G′ was recovered under a small strain (5%) within few seconds. The obtained HA-BOR showed self-healing properties, (FIG. 3). The characteristics of the resulting samples are shown in Table 3. The results show that HA-PBA gives a viscoelastic behaviour, whereas HA-BOR gives a gel with a number of different molecular weights.

TABLE 3
Characteristics of obtained samples ([PS] = 15 g/L).

| HA | DS HA | Mw HA | G′ 1 Hz | G″ 1 Hz | Rheological |
|---|---|---|---|---|---|
| derivative | derivative | (kg/mol) | (Pa) | (Pa) | behavior |
| HA-BOR | 0.1 | 100 | 0.043 | 0.44 | Viscous |
| HA-BOR | 0.1 | 500 | 160 | 38 | Gel |
| HA-BOR | 0.2 | 500 | 204 | 63 | Gel |
| HA-BOR | 0.1 | 600 | 330 | 108 | Gel |
| HA-BOR | 0.2 | 600 | 800 | 210 | Gel |
| HA-BOR | 0.1 | 1000 | 45 | 29 | Viscoelastic |
| HA-BOR | 0.2 | 1000 | 198 | 78 | Gel |
| HA-PBA | 0.15 | 600 | 5.65 | 5.89 | Viscoelastic |
| Native HA | — | 500 | 0.05 | 1.3 | Viscous |
| Native HA | — | 500 | 0.1 | 1.96 | Viscous |
| Native HA | — | 600 | 2 | 8 | Viscous |
| Native HA | — | 1000 | 27 | 33 | Viscoelastic |

### Example 5: Synthesis of Pentenoate-Modified HA

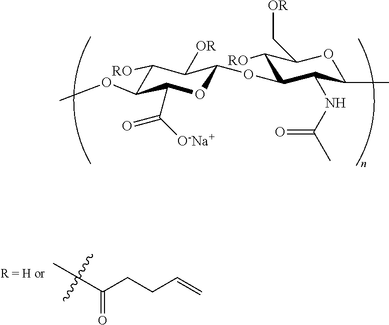

HA (1 g, 2.5 mmol, M_{W}=100 kg/mol) was dissolved in ultrapure water (50 mL) under continuos stirring overnight at 4° C. DMF (33 mL) was then added dropwise in order to have a water/DMF ratio of (3/2, v/v). 4-pentenoic anhydride (0.454 g, 2.5 mmol) was added while maintaining the pH between 8 and 9 by adding 1 M NaOH for at least 4 h. The reaction was kept at 4° C. under stirring for one night. The product was purified by diafiltration with ultrapure water and was recovered by freeze-drying. The degree of substitution (DS) of HA-pentenoate was found to be 0.18±0.01 by ¹H NMR. A yield of 49% was calculated considering its DS.

¹H NMR (400 MHz, D₂O) δ_{H} (ppm) 4.71 (H-1 from N-acetylglucosamine unit), 4.53 (H-1 from glucuronic acid), 4.13-3.2 (H-2, H-3, H-4, H-5, H-6 protons of HA), 2.1 (CH₃—CO from HA), 6.0 (m, 1H, CH═CH2), 5.18 (m, 2H, CH═CH₂), 2.62 (m, 2H, CH₂—C═O), 2.45 (m, 2H, OCCH₂—CH₂).

### Example 6: Synthesis of HA-Maltose

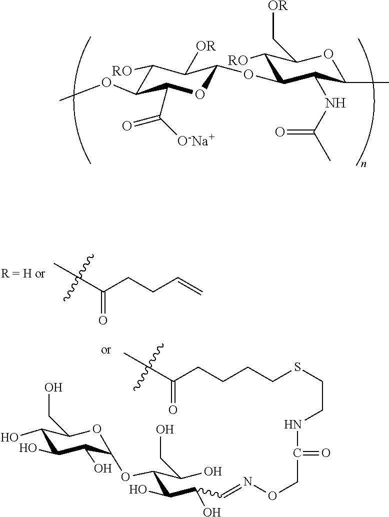

### a. Maltose-Disulfide

To an aqueous solution of maltose (0.25 g, 0.694 mmol) in 25 mL of ultrapure water at room temperature, O-(carboxymethyl)hydroxylamine hemihydrochloride (0.0768 g, 0.694 mmol) was added. The pH was adjusted to 4.8 using 0.5 M NaOH. The reaction mixture was stirred for 24 hours at room temperature and then, was neutralized to pH 7 by addition of 0.5 M NaOH. The maltose-COOH derivative was then recovered by freeze-drying without further purification as a white powder (46 mol % of maltose-COOH/maltose). To a solution of maltose-COOH (0.25 g, 0.622 mmol) in dry DMF (50 mL), hydroxybenzotriazole (HOBt) (0.1875 g, 1.39 mmol), diisopropylcarbodiimide (DIC) (0.3483 g, 2.8 mmol) and cystamine dihydrochloride (0.094 g, 0.42 mmol) were successively added. The resulting mixture was stirred overnight at room temperature under nitrogen. After evaporation of most of the solvent, the residual syrup was poured dropwise into acetone (500 mL) under stirring. The white precipitate was collected by filtration, washed three times with acetone and dried to give the desired maltose-disulfide in 60% yield (0.295 g).

¹H NMR (400 MHz, D₂O) δ_{H} (ppm) 7.75 (1H, anomeric Hβ from linked glucose unit, N═CH_{β}—), 7.13 (1H, anomeric Hα from linked glucose unit, N═CH_{α}—), 5.4 (1H, anomeric H from pendant glucose unit of maltose), 5.19 (1H, anomeric Hα from linked glucose unit), 5.14 (1H, anomeric H from pendant glucose unit of maltose-disulfide), 4.7 (1H, anomeric Hβ from pendant glucose unit), 4.66 (2H, N—O—CH₂), 4.6 (1H, N═CH_{α,β}—CH(OH) from linked glucose group), 3.4-4.2 (8H, H-3, H-4, H-5, H-6 from linked and pendant glucose groups), 2.95 (4H, NH—CH₂—CH₂).

### b. HA-Maltose

The first step consisted in reducing the disulfide bond of maltose-disulfide. Thus, to an aqueous solution of this derivative (0.2 g, 0.211 mmol) in 4 mL of degassed phosphate buffered saline (PBS) pH 7.4 at room temperature, a solution of TCEP (91 mg, 0.317 mmol) in 1 mL of degassed PBS was added and the pH was adjusted to 5-5.5. The mixture was stirred for 15 min under nitrogen at room temperature to give maltose-SH. The pH was adjusted to 7.4 using 0.5 M NaOH and the mixture was added to HA-pentenoate solubilized in PBS in the presence of Irgacure 2959 (0.1%, w/v) as a photoinitiator. The grafting of maltose-SH moieties was performed under UV radiation (λ=365 nm, at 20 mW/cm² for 15 min). The product was purified by diafiltration with ultrapure water and was recovered by freeze-drying (80%). The degree of substitution (DS) of HA-maltose was found to be 0.1±0.01 by ¹H NMR.

¹H NMR (400 MHz, D₂O) δ_{H} (ppm) 4.55 (H-1 from N-acetylglucosamine unit), 4.25 (H-1 from glucuronic acid), 3.9-3.1 (H-2, H-3, H-4, H-5, H-6 protons of HA), 1.85 (CH₃—CO from HA), 1.52 (m,2H,CH₂—CH₂—CH₂—S), 1.62 (m,2H,CH₂—CH₂—CH₂—S), 2.35 (m, 2H, OC—CH₂) 2.63 (m,2H, CH₂—CH₂—CH₂—S), 2.82 (m,2H, S—CH₂—CH₂—NH), 7.63 (m, 1H, H anomer of maltose).

### Example 7: Synthesis of HA-Lactobionic

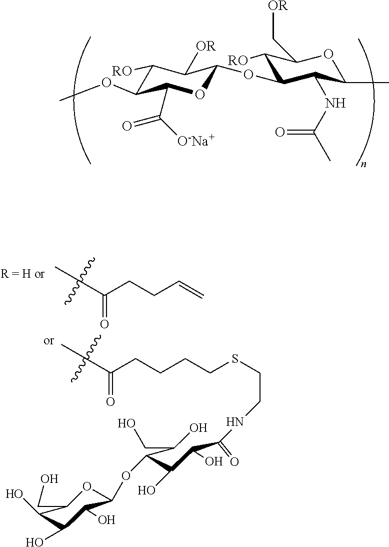

### a. Lactobionic-Disulfide

To a solution of lactobionic acid (0.5023 g, 1.39 mmol) in dry DMF (50 mL), hydroxybenzotriazole (HOBt) (0.3768 g, 2.79 mmol), diisopropylcarbodiimide (DIC) (0.705 g, 5.56 mmol) and cystamine dihydrochloride (0.141 g, 0.63 mmol) were successively added. The resulting mixture was stirred overnight at room temperature under nitrogen. After evaporation of most of the solvent, the residual syrup was poured dropwise into acetone (500 mL) under stirring. The white precipitate was collected by filtration, washed three times with acetone and dried to give the desired lactobionic-disulfide in 29% yield (0.2362 g).

### b. HA-Lactobionic

A first step of reduction of the disulfide bond of the lactobionic-disulfide derivative (0.2 g, 0.211 mmol) dissolved in 1 mL of degassed PBS was performed by adding TCEP (91 mg, 0.317 mmol) in 1 mL of degassed PBS, with pH adjusted to 5-5.5. The mixture was stirred for 15 min under nitrogen at room temperature to give lactobionic-SH. The pH was adjusted to 7.4 using 0.5 M NaOH and the mixture was added to HA-pentenoate solubilized in PBS in the presence of Irgacure 2959 (0.1%, w/v) as a photoinitiator. The grafting of lactobionic-SH moieties was performed under UV radiation (λ=365 nm, at 20 mW/cm² for 15 min). The product was purified by diafiltration with ultrapure water and was recovered by freeze-drying (60%). The degree of substitution (DS) of HA-lactobionic was found to be 0.2±0.01 by ¹H NMR.

### Example 8: Synthesis of HA-Fructose

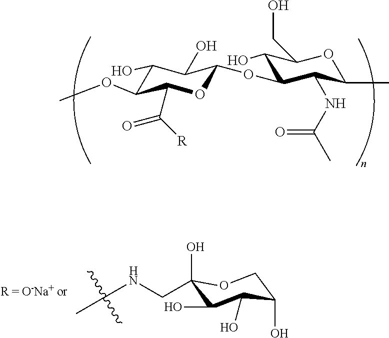

1-amino-1-deoxy-D-fructose hydrochloride (0.0121 g, 0.056 mmol) dissolved in 1 mL of ultrapure water was added to a solution of native HA (0.15 g, 0.374 mmol) in a mixture of water/DMF (3/2, v/v) in the presence of 4-(4,6-dimethoxy-1,3,5-triazin-2-yl)-4-methylmorpholinium chloride (DMTMM) (0.1035 g, 0.374 mmol) as an amine-acid coupling agent. The pH was adjusted to 6.5 using 0.5 M HCl or NaOH and the reaction was kept under stirring at room temperature for 24 h. The product was purified by diafiltration with ultrapure water and was recovered by freeze-drying. The degree of substitution (DS) of HA-fructose was determined by ¹³C NMR (DS_{NMR}=0.15±0.01), and was also estimated from the reaction kinetics performed using 2,4,6-Trinitrobenzene Sulfonic Acid (DS_{TNBS}=0.14). A yield of 84% was determined for HA-fructose (considering its DS_{NMR}).

¹H NMR (400 MHz, D₂O) δ_{H} (ppm) 4.62 (H-1 from N-acetylglucosamine unit), 4.46 (H-1 from glucuronic acid), 4.05-3.2 (18H, H-2, H-3, H-4, H-5, H-6 protons of HA and of fructose moieties), 2.02 (CH₃—CO from HA).

### Example 9: Synthesis of HA-Sorbitol

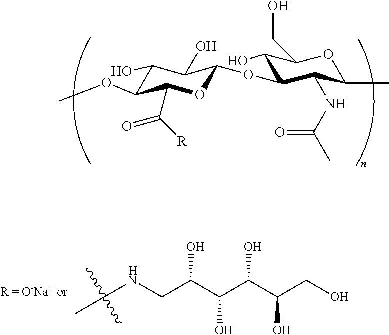

1-amino-1-deoxy-D-sorbitol hydrochloride (D-glucamine) (0.0088 g, 0.05 mmol) dissolved in 1 mL of ultrapure water was added to a solution of native HA (0.1305 g, 0.325 mmol) in ultrapure water in the presence of 4-(4,6-dimethoxy-1,3,5-triazin-2-yl)-4-methylmorpholinium chloride (DMTMM) (0.09 g, 0.325 mmol) as an amine-acid coupling agent. The pH was adjusted to 6.5 using 0.5 M HCl or NaOH and the reaction was kept under stirring at room temperature for 164 h. The product was purified by diafiltration with ultrapure water and was recovered by freeze-drying. The degree of substitution (DS) of HA-sorbitol was determined by ¹³C NMR (DS_{NMR}=0.15±0.1), and was also estimated from the reaction kinetics performed using 2,4,6-Trinitrobenzene Sulfonic Acid (DS_{TNBS}=0.1). A yield of 76% was determined for HA-sorbitol (considering its DS_{NMR}).

¹H NMR (400 MHz, D₂O) δ_{H} (ppm) 4.68 (H-1 from N-acetylglucosamine unit), 4.51 (H-1 from glucuronic acid), 4.1-3.3 (19H, H-2, H-3, H-4, H-5, H-6 protons of HA and of sorbitol moieties), 2.07 (CH₃—CO from HA).

### Example 10: Preparation of HA-BOR/HA-Polyol Gel

Solutions of HA-BOR and of the HA-polyol derivatives (HA-fructose or HA-sorbitol) were prepared at 15 g/L in 0.01 M HEPES buffer containing 0.15 M NaCl pH 7.4, and were kept under stirring overnight at 4° C. Combinations of HA-BOR/HA-polyol derivative, were prepared by mixing a solution containing HA-BOR with a solution containing a HA-polyol derivative at physiological pH, at a total polymer concentration of 15 g/L and with BOR/polyol molar ratio of 1/1.

Results:

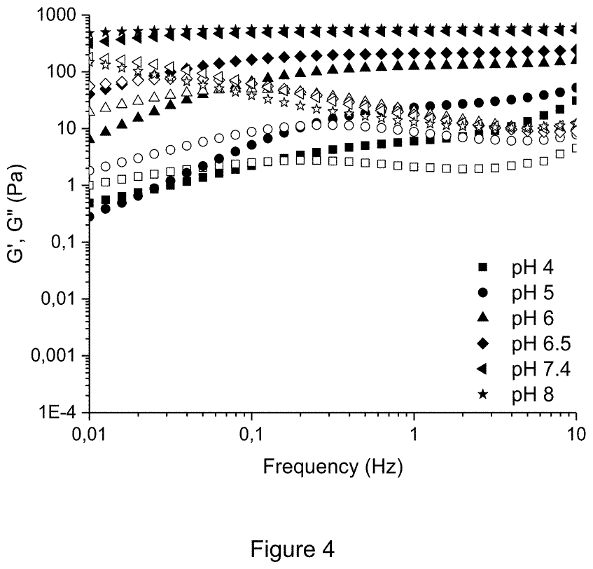

When gels were formed quasi-instantaneously upon mixing HA-BOR solution with a solution of a HA-polyol derivative. Characteristics of the resulting HA-BOR/HA-polyol mixtures are summarized in Table 4. A rheological analysis of HA-BOR/HA-fructose is shown in FIG. 4.

TABLE 4
Characteristics of HA-BOR/HA-Polyol hydrogel ([PS] = 15 g/L).

| DS HA- | | DS HA- | | G′ | G″ | |
|---|---|---|---|---|---|---|
| BOR | HA-polyol | polyol | Mw HA | 1 Hz | 1 Hz | Rheological |
| derivative | derivative | derivative | (kg/mol) | (Pa) | (Pa) | behavior |
| 0.16 | HA-maltose | 0.12 | 75 | 34 | 20 | Viscoelastic |
| 0.16 | HA-fructose | 0.15 | 75 | 500 | 17 | Gel |
| 0.16 | HA-sorbitol | 0.15 | 75 | 250 | 125 | Viscoelastic |
| 0.12 | HA-fructose | 0.15 | 100 | 490 | 7 | Gel |
| 0.11 | HA-fructose | 0.08 | 600 | 250 | 80 | Gel |

### Example 11: Doubly CL HA Gels

Two methods were employed to synthesize doubly crosslinked hyaluronic acid gels: i) cross-linking of a HA1000-BOR derivative and of a HA1000-fructose/HA1000-PBA mixture by reaction of HA hydroxyl groups with BDDE (method no. 1); ii) grafting of BOR or PBA or fructose moieties on HA-BDPE gel particles by a peptide-like coupling reaction (method no. 2). The products synthesized by the method no. 2 were purified by diafiltration (UF) with ultrapure water and were recovered by freeze-drying.

Results:

Table 5 summarizes the syntheses of doubly crosslinked gels by method no. 2.

TABLE 5
Summary of the syntheses of doubly crosslinked gels by method no. 2.

| | | Functional | | | | |
|---|---|---|---|---|---|---|
| | DMTMM/HA | molecule/HA | Membrane UF | | | Yield |
| Derivative | molar ratio | molar ratio | MWCO (kDa) | DS_{NMR} | DS_{TNBS} | (%)^{d} |
| HA-BDPE/BOR | 1 | 0.16 | 30 | 0.12^{b} | 0.14 | ≈ 100^{e} |
| HA- | 1 | 0.15 | 30 | 0.1^{c} | 0.11 | ≈ 100^{e} |
| BDPE/fructose | | | | | | |
| HA-BDPE/BOR | 1 | 0.15 | 3 | 0.08^{b} | 0.12 | ≈ 100^{e} |
| alkaline | | | | | | |
| treatment^{a} | | | | | | |
| HA-BDPE/PBA | 1 | 0.15 | 3 | 0.11^{b} | 0.15 | ≈ 100^{e} |
| alkaline | | | | | | |
| treatment^{a} | | | | | | |
| HA-BDPE | 1 | — | 30 | — | — | — |
| control | | | | | | |

^{a}Alkaline treatment sequential to peptide coupling: 0.25 M NaOH (pH ≥ 13) at RT for 1 h.
^{b}DS by ¹H NMR after enzymatic degradation: 10% of accuracy.
^{c}DS by ¹³C NMR after enzymatic degradation: 20% of accuracy.
^{d}Yield calculated considering the DS_{NMR} of the HA derivative.
^{e}Imprecision of values probably related to variations of the concentration of HA in the initial syringes of HA-BDPE gel particles.

For clarity, the samples prepared following method no. 1, were named J1-3, whereas the ones obtained from method no. 2 were named T1-5. Scheme 1 illustrates the preparation of samples T1-5, by simply solubilizing modified HA-BDPE gel particles as a powder in a 1 mM phosphate/0.9% NaCl buffer pH 7.4 at a polymer concentration of 20 g/L. J1-3 samples were analyzed under the same conditions, and were recovered as hydrogels at the end of the cross-linking reaction of HA1000 derivatives using BDDE. Table 6 summarizes the rheological properties of these samples, measured by experiments of dependence on frequency of the rheological moduli. The results show that the HA-BDPE/BOR gel has the highest G′ and that it has improved properties after alkaline treatment than does HA-BDPE/PBA.

TABLE 6
Doubly crosslinked gels prepared by method no. 1 and 2 and their
characterization by rheology.

| | | UF | | Rheo- | | |
|---|---|---|---|---|---|---|
| | | MWCO | | logical | G′ 1 Hz | G″ 1 Hz |
| Ref. | Sample | (kDa) | DS_{NMR}^{a} | behavior | (Pa) | (Pa) |
| J1 | HA/BDPE control | — | — | Gel | 909 | 132 |
| J2 | HA-PBA/HA- | — | 0.14/0.1 | Gel | 32.5 | 4.6 |
| | fructose/BDPE | | | | | |
| J3 | HA-BOR/BDPE | — | 0.1 | Gel | 680 | 170 |
| T1 | HA-BDPE control | 30 | — | Gel | 225 | 63.4 |
| T2 | HA-BDPE/BOR | 30 | 0.12 | Gel | 1930 | 340 |
| T3 | HA-BDPE/BOR + | 30 | 0.12/0.1 | Gel | 516 | 103 |
| | HA-BDPE/fructose | | | | | |
| T4 | HA-BDPE/BOR | 3 | 0.08 | Gel | 1320 | 250 |
| | alkaline treatment^{b} | | | | | |
| T5 | HA-BDPE/PBA | 3 | 0.11 | Gel | 403 | 76 |
| | alkaline treatment^{b} | | | | | |

^{a}DS of BOR- or PBA- or fructose-modified HA.
^{b}Alkaline treatment sequential to peptide coupling: 0.25 M NaOH (pH ≥ 13) at RT for 1 h.

### Example 12: Self Healing Properties of Obtained Gels

The variation of G′ and G″ as a function of time immediately after injection through a 27 gauge needle of HA-BDPE/BOR and HA-BDPE control was investigated. Gels were prepared in 1 mM sodium sulphate/0.9% NaCl buffer pH 7.4, at a [PS]=20 g/L.

Results:

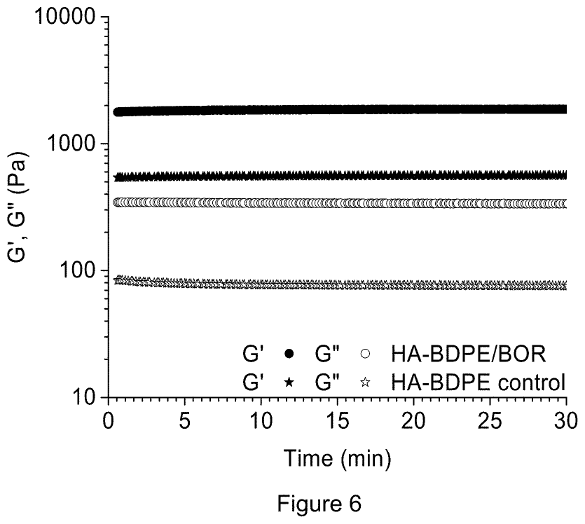

The hydrogel exhibited self-healing properties. Consequently, it can be injected as preformed solids, because the solid gel can manage external damages and repair itself under a proper shear stress. Due to fast gelation kinetics after extrusion/injection, they recover their solid form immediately. As an example, FIG. 6 shows the variation of G′ and G″ as a function of time immediately after injection of the HA-BDPE/BOR and HA-BDPE control gels through a 27 gauge needle. From this Figure, it can be seen that the three samples recovered into a solid gel quasi-instantaneously.

1-51. (canceled)

52. Glycosaminoglycans crosslinked by a first and a second linkage, wherein
a) said first linkage comprises two ether bonds, one bond formed with a hydroxyl group of each of a first glycosaminoglycan and a second glycosaminoglycan; and
b) said second linkage is via an alkoxyboronate ester anion formed between a boronate hemiester grafted to the first glycosaminoglycan and a diol function of the second glycosaminoglycan,
wherein said diol function may be a backbone diol function or a diol portion of a diol functional moiety grafted to said second glycosaminoglycan.

53. Crosslinked glycosaminoglycans according to claim 52, wherein said second linkage is defined in Formula (I) 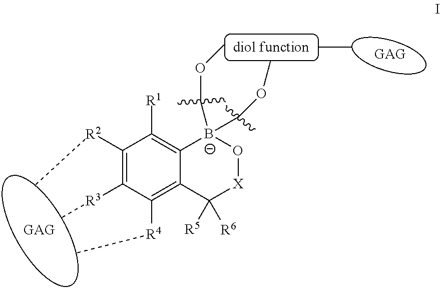
wherein
R¹ is selected from H, F, Cl, NO₂, C₁-C₃alkyl, C₁-C₃haloalkyl and C₁-C₃alkoxy;
R², R³ and R⁴ are independently selected from H, F, Cl, C₁-C₃haloalkyl, NO₂, C₁-C₃alkoxy, C₁-C₃alkyl and a linker, said linker binding covalently to said first glycosaminoglycan;
X is selected from CHR⁷ and a bond;
R⁵, R⁶ and R⁷ are independently selected from H, C₁-C₄alkyl, C₃-C₆cycloalkyl, phenyl, and a five- to six-membered heteroaromatic ring comprising 1 to 3 heteroatoms selected from O, N and S; and
wherein one of R², R³ and R⁴ is a linker.

54. Crosslinked glycosaminoglycans according to claim 52, wherein said glycosaminoglycans are hyaluronic acid.

55. Crosslinked glycosaminoglycans according to claim 53, wherein R² is a linker.

56. Crosslinked glycosaminoglycans according to claim 53, wherein said linker is
—NR⁹—Y— and forms an amide bond with said first glycosaminoglycans, wherein R⁹ is selected from hydrogen, C₁-C₃alkyl and C₁-C₃fluoroalkyl; and
Y is a bond or an unsubstituted C₁-C₆alkylene.

57. Crosslinked glycosaminoglycans according to claim 53, wherein
R¹, R³ and R⁴ are independently selected from H, F, OCH₃, CF₃ and CH₃;
R² is a linker;
said linker is —HN—Y— and forms an amide bond with said first glycosaminoglycan;
Y is a bond or an unsubstituted C₁-C₃alkylene;
X is a bond or CH₂; and
R⁵ and R⁶ are independently selected from H and C₁-C₃alkyl.

58. Crosslinked glycosaminoglycans according to claim 52, wherein said boronate hemiester is selected from 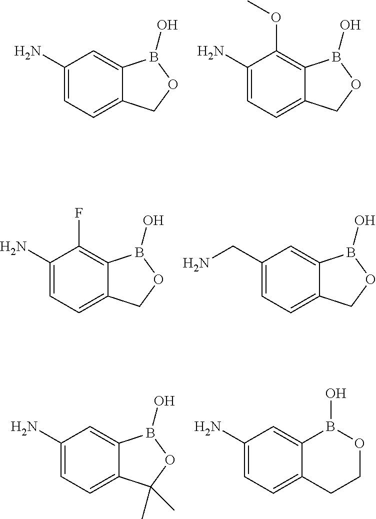
wherein the boronate hemiester is grafted to said first glycosaminoglycan by that the —NH₂ group of the boronate hemiester forms an amide with a backbone carboxylate group of said first glycosaminoglycan.

59. Crosslinked glycosaminoglycans according to claim 52, said second linkage having a structure of Formula (II)

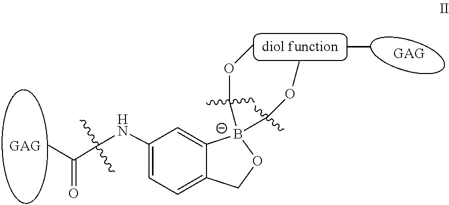

60. Crosslinked glycosaminoglycans according to claim 52, wherein said diol function is a backbone diol function.

61. Crosslinked glycosaminoglycans according to claim 52, wherein said diol function is a diol portion of a diol functional moiety grafted to said second glycosaminoglycan,
wherein said diol portion is selected from a monosaccharide, a disaccharide and an alditol or a derivative thereof, or wherein said diol portion is selected from maltose, fructose, lactose and sorbitol or a derivative thereof.

62. A method of crosslinking glycosaminoglycans, comprising the steps of:
forming a linkage comprising two ether bonds, one bond formed with a hydroxyl group of each a first and a second glycosaminoglycan;
grafting said first glycosaminoglycan with a boronate hemiester and crosslinking said first glycosaminoglycan with said second glycosaminoglycan by forming an alkoxyboronate ester anion linkage between the boronate hemiester of said first glycosaminoglycan and a diol function of said second glycosaminoglycan, wherein said diol function may be a backbone diol function or a diol portion of a diol functional moiety grafted to said second glycosaminoglycan.

63. A method according to claim 62, wherein said boronate hemiester is a compound of Formula (III), 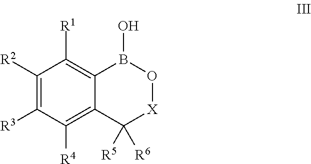
wherein
R¹ is selected from H, F, Cl, NO₂, C₁-C₃alkyl, C₁-C₃haloalkyl and C₁-C₃alkoxy;
R², R³ and R⁴ are independently selected from H, F, Cl, C₁-C₃haloalkyl, NO₂, C₁-C₃alkoxy, C₁-C₃alkyl and a linker binding covalently to said first glycosaminoglycan;
X is selected from CHR⁷ and a bond; and
R⁵, R⁶ and R⁷ are independently selected from H, C₁-C₄alkyl, C₃-C₆cycloalkyl, phenyl, and a five- to six-membered heteroaromatic ring comprising 1 to 3 heteroatoms selected from O, N and S,
wherein one of R², R³ and R⁴ is a linker.

64. A method according to claim 62, wherein said first and said second glycosaminoglycans are hyaluronic acid.

65. A method according to claim 63, wherein R² is a linker.

66. A method according to claim 63, wherein said linker is HR⁹N—Y— and forms an amide bond with said first glycosaminoglycan, wherein R⁹ is selected from hydrogen, C₁-C₃alkyl and C₁-C₃fluoroalkyl; and
Y is a bond or an unsubstituted C₁-C₆alkylene.

67. A method according to claim 63, wherein
R¹, R³ and R⁴ are independently selected from H, F, OCH₃, CF₃ and CH₃;
R² is a linker;
said linker is H₂N—Y— and forms an amide bond with said first glycosaminoglycan;
Y is a bond or an unsubstituted C₁-C₃alkylene;
X is a bond or CH₂; and
R⁵ and R⁶ are independently selected from H and C₁-C₃alkyl.

68. A method according to claim 62, wherein said boronate hemiester is selected from 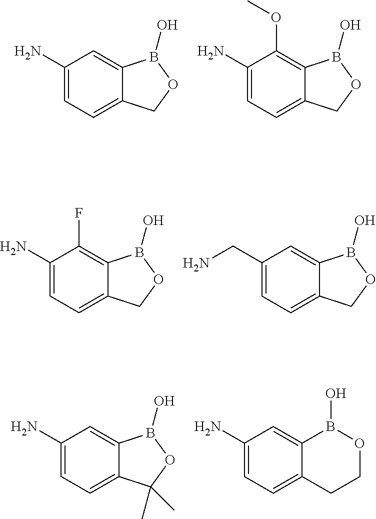
wherein the boronate hemiester is grafted to said first glycosaminoglycan by that the —NH₂ group of the boronate hemiester forms an amide with a backbone carboxylate group of said first glycosaminoglycan.

69. A method according to claim 62, wherein said diol function is a backbone diol function.

70. A method according to claim 62, wherein said diol function is a diol portion of a diol functional moiety grafted to said second glycosaminoglycan,
wherein said diol portion is selected from a monosaccharide, a disaccharide and an alditol or a derivative thereof, or selected from maltose, fructose, lactose and sorbitol or a derivative thereof.

71. Polymer composition comprising crosslinked glycosaminoglycans according to claims 52 and an aqueous buffer.
